(19) United States
(12) Patent Application Publication
(10) Pub. No.: US 2008/0292580 A1
(43) Pub. Date: Nov. 27, 2008

(54) Treatment of Inflammatory Diseases

(75) Inventors: Gareth Ackland, Emsworth (GB); Alexander V. Gourine, London (GB)

Correspondence Address: SPECKMAN LAW GROUP PLLC, 1201 THIRD AVENUE, SUITE 330, SEATTLE, WA, 98101, US

(73) Assignee: UNIVERSITY COLLEGE LONDON HOSPITALS NHS FOUNDATION, London (GB)

(21) Appl. No.: 11/722,784

(22) PCT Filed: Dec. 23, 2005
(86) PCT No.: PCT/GB05/05070

§ 371 Date: Jul. 28, 2008

(30) Foreign Application Priority Data
Dec. 23, 2004 (GB) 0428218.2

(51) Int. Cl. A61K 31/765; A61P 29/00

(52) U.S. Cl. 424/78.38

(57) ABSTRACT

An aqueous solution or at least one polyether polyol in a fluid for administration to a mammal, such as a human being, wherein the at least one polyether polyol has a molecular weight of from 200 to 4000 and is dissolved in the aqueous fluid in a concentration of from 0.01 to 10 mg/ml, preferably from 1 to 10 mg/ml.

The present invention relates to a medicament for the treatment of inflammatory diseases and to the use of such a medicament for the treatment of inflammatory diseases.

The hallmark of acute medical and surgical emergencies is the generation of massive quantities of inflammatory mediators, initiated by cytokines. Across a range of both acute and chronic diseases, high levels of inflammation result in premature death or costly morbidity. Currently, 215,000 deaths from sepsis (inflammation generated by infection) occur in the US alone with associated healthcare costs of $16.7 billion—in total, critically ill patients consume 0.6-1.0% of US GDP per annum. Similarly, in the UK sepsis accounts for up to 11% of all hospital or intensive care admissions. The incidence of sepsis is projected to increase by 1.5% per annum. Despite intensive research efforts over the past 25 years, mortality and morbidity from sepsis, massive trauma or major surgery, remains high. Sepsis in the intensive care unit typically results in 50% mortality. A key, universally accepted component of treating these common clinical problems is the administration of fluid. Despite being a central part of many medical management strategies, little research has been undertaken to add anti-inflammatory properties to the fluids that are in any event always administered to critically ill patients.

It is known in the art to treat haemorrhagic shock using a variety of different fluid compositions. However, haemorrhagic shock is quite distinct from septic shock, and there is no indication that compositions known in the art to treat haemorrhagic shock would be effective to treat septic shock.

In a paper entitled “Survival in a rat model of lethal hemorrhagic shock is prolonged following resuscitation with a small volume of a solution containing a drag-reducing polymer derived from aloe vera”, Carlos A. Macias et al., Shock, Vol. 22, No. 2, pp. 151-156, 2004, the use of resuscitation solutions containing a drag-reducing polymer derived from aloe vera is disclosed for treating rats in a laboratory model of haemorrhage. However, the experimental data showed rather equivocal results with regard to the results using the drag-reducing polymer as compared to saline as a control. Also, the results were a laboratory model of haemorrhage. There is no result directly addressing the treatment of acute or chronic inflammatory conditions.

As another example, WPI Accession No 1994-125274/15 & SU 1635330 discloses a solution based on a high molecular weight polyethylene glycol (PEG), having a molecular weight of 15,000 to 20,000, administered in a concentration, 10-20 g/l, in an experimental model of haemorrhagic shock. The mechanism of action proposed by the authors, stated to be enhancement of haemodynamic effects and rheological properties of the blood, has no direct relevance to any reduction of the systemic inflammatory process relevant to the treatment of septic shock.

As a further example, a paper in Biorheology (2004) Vol 41, pp 53-64, entitled “Blood soluble drag-reducing polymers prevent lethality form hemorrhagic shock in acute animal experiments” by Kameneva et al discloses the use of polyethylene glycol, 3500 kDa, as a drag reducing polymer in a model of haemorrhagic shock. However, the authors demonstrated the ineffectiveness of low molecular weight polyethylene glycol in their experimental model and concluded that the effect of polyethylene glycol is not related to its chemical properties. The mechanism of action suggested by the authors is related to drag-reducing properties of high molecular weight polyethylene glycol, which, in contrast to low molecular weight polyethylene glycol, has been shown to reduce flow resistance. This proposed mechanism has no relevance to any reduction of the systemic inflammatory process which is relevant to the treatment of septic shock.

Polyether polyol, in particular polyether glycol, and most particularly polyethylene glycol, is a widely used substance for both household and industrial purposes. It possesses some remarkable properties that have been explored in several laboratory-based scenarios, although the mechanisms through which polyethylene glycol acts under different experimental conditions are unclear. Polyethylene glycol improves function in experimental transplant organs (J P Faure et al, American Journal of Transplantation 2004 vol 4; 495-504) reverses experimental spinal cord injury (R Borgens and R Shi, FASEB J. vol 14, 27-35, 2000) and protects against experimentally-induced colonic cancer (D E Corpet et al, Carcinogenesis vol. 20 no. 5 pp. 915-918, 1999).

It is also known in the art to employ polyethylene glycol as a vehicle for therapeutic agents in a process known in the art as “PEGylation”.

For example, a paper in the Journal of Surgical Research (1995), Vol 59, pp 153-158, entitled “PEG-BP-30 Monotherapy Attenuates the Cytokine-Mediated Inflammatory Cascade in Baboon Escherichia coli Septic Shock . . . ” by Espat et al discloses the effects of soluble tumour necrosis factor receptors linked to polyethylene glycol used in a “PEGylation” technique to reduce immunogenecity and clearance and increase plasma half-life of the soluble receptor. Experimental design of this study, like of the vast majority of other studies involving peptides linked to polyethylene glycol, is faulty. First, this study fails to consider the most important control, namely determining the effect of polyethylene glycol per se. Second, PEG-BP-30 was given before induction of sepsis, invalidating clinical significance of the study. Therefore, based on this study no conclusion can be made regarding the potential use of polyethylene glycol to treat inflammatory conditions and sepsis.

As another example, a paper in Clinical Science (2004), Vol 107, pp. 263-272, entitled “Plasma expansion by polyethylene-glycol-modified albumin” by Assaly et al discloses the effects of albumin linked to polyethylene glycol in a “PEGylation” technique to modify albumin. Experimental design of this study is also faulty: the most important control, namely administration of polyethylene glycol per se is missing. Similarly to the study discussed above PEG-albumin treatments were given before induction of sepsis, invalidating any clinical significance of the study. Thus, no conclusions about the potential use of polyethylene glycol to treat inflammatory conditions and sepsis can be made based on this paper.

FR-A-2316923 discloses complex solutions comprising a number of components (urethane, ethylene glycol, etc) including high molecular weight (6000) polyethylene glycol in a high concentration (2.5%). There is no evidence given that the beneficial effect of this solution is related to the properties of polyethylene glycol.

DE-A-10204696 discloses the use of polyethylene glycol in a nasal spray (comprising several other substances) to treat viral infections causing coughs and sneezes.

WO-A-2004/047778 discloses a solution based on a high molecular weight, at least 5,000 daltons, polyethylene glycol given in a very high concentration in an experimental model of microbe-mediated epithelial disorders. In the examples disclosed, the pathogen (Pseudomonas aeruginosa) was administered mixed with a polyethylene glycol solution. It is proposed that high molecular weight, high concentration polyethylene glycol prevents contact of pathogens with the epithelial surface, which is not relevant to reduction of the systemic inflammatory process. The disclosure has no relevance to the treatment of acute polymicrobial sepsis after the insult, when the pathological components may already be completely cleared.

Furthermore, polyethylene glycols are known for use as bowel purgatives in common clinical practice.

None of these prior disclosures addresses the treatment of major acute and chronic inflammatory conditions, in particular for surgical and medical emergencies, and for chronic inflammatory disease.

There is a need for improved treatment of major acute and chronic inflammatory conditions, in particular for surgical and medical emergencies, and for chronic inflammatory disease.

There is also a need for such a treatment that can attenuate and/or resolve major acute and chronic inflammatory conditions.

There is further a need for such a treatment that can be administered readily and effectively.

There is yet further a need for such a treatment that can be administered using as active component a readily available compound, known to be safe for use both in foods and in drugs for administration to humans.

The present invention at least partially aims to meet at least one of those needs.

In a first aspect, the present invention provides an aqueous solution of at least one polyether polyol in an aqueous fluid for administration to a mammal, wherein the at least one polyether polyol has a molecular weight of from 200 to 4000 and is dissolved in the aqueous fluid in a concentration of from 0.01 to 10 mg/ml.

In a second aspect, the present invention provides an aqueous solution of at least one polyether polyol in an aqueous fluid for administration to a mammal for use as a medicament, wherein the at least one polyether polyol has a molecular weight of from 200 to 4000 and is dissolved in the aqueous fluid in a concentration suitable for the treatment of inflammatory disease, preferably from 0.01 to 10 mg/ml.

In a third aspect, the present invention provides the use of an aqueous solution of at least one polyether polyol, the at least one polyether polyol having a molecular weight of from 200 to 4000 and being dissolved in the aqueous solution in a concentration suitable for the treatment of inflammatory disease, preferably from 0.01 to 10 mg/ml, for administration to a mammal for the manufacture of a medicament for treating inflammatory disease.

In a fourth aspect, the present invention provides the use of a composition comprising at least one polyether polyol and an aqueous solution, the at least one polyether polyol having a molecular weight of from 200 to 4000 and being dissolved in the aqueous solution in a concentration suitable for the treatment of inflammatory disease, preferably from 0.01 to 10 mg/ml, for administration to a mammal for the manufacture of a combined preparation for the simultaneous, separate or sequential use as a medicament for treating inflammatory disease.

In a fifth aspect, the present invention provides a composition comprising an aqueous solution of at least one polyether polyol having a molecular weight of from 200 to 4000 and dissolved at a concentration of from 0.01 to 10 mg/ml for administration to a human being as a combined preparation for use as a medicament, in particular for treating inflammatory disease.

In each of these aspects, preferably the concentration of the at least one polyether polyol in the aqueous solution is from 1 to 10 mg/ml.

In a sixth aspect, the present invention provides a medicament for administration to a mammal, the medicament comprising at least one polyether polyol in aqueous solution, the at least one polyether polyol consisting of the sole active therapeutic constituent of the medicament, wherein the at least one polyether polyol has a molecular weight of from 200 to 4000 and is dissolved in an aqueous fluid selected from an aqueous solution of one or more crystalloids, an aqueous colloid suspension of one or more colloids, or a mixture of such an aqueous solution of one or more crystalloids and such a colloid suspension of one or more colloids.

Preferably the concentration of the at least one polyether polyol in the aqueous solution is from 0.01 to 10 mg/ml, more preferably from 1 to 10 mg/ml.

In accordance with these aspects of the present invention, the polyether polyol may comprise a single polyether polyol, or may comprise a mixture of at least two polyether polyols. The polyether polyol is preferably a polyether glycol, most preferably polyethylene glycol. The polyethylene glycol preferably has a molecular weight of from 200 to 1000.

The invention most preferably has medical use for treating human beings, although it may also have veterinary use for treating other mammals, such as domestic pets (cats, dogs, etc.), horses, farm animals (cattle, etc.).

The aqueous fluid for administration to a human being is a medical-grade liquid, such as an aqueous intravenous resuscitation fluid or an aqueous fluid for intrathecal or intraperitoneal therapy which preferably comprises an aqueous solution of one or more crystalloids, an aqueous colloid suspension of one or more colloids, or a mixture of such an aqueous solution of one or more crystalloids and such a colloid suspension of one or more colloids. Preferred aqueous crystalloid solutions include Ringer's lactate solution, compound sodium lactate solution (for example comprising sodium 131 mmol/l, potassium 5 mmol/l, calcium 2 mmol/l, chloride 111 mmol/l, lactate 29 mmol/l; pH 6-7; osmolarity 278 mOsmol/l) and normal saline solution (for example 0.9% sodium chloride in water), or mixtures of two or more of these solutions. Preferred aqueous colloid suspensions include succinylated gelatine suspended in, for example 0.9% saline solution and starch based colloid preparations (including hydroxyethylated starch) in aqueous suspension. The preferred crystalloid-colloid solutions include one or more aqueous crystalloid solutions in admixture with one or more aqueous colloid suspensions.

The at least one polyether polyol is dissolved in the aqueous fluid for administration to a human being in a concentration (with respect to the volume of the initial aqueous fluid) of from 0.01 to 10 mg/ml, more preferably from 1 to 10 mg/ml.

In a seventh aspect, the present invention provides a method of treating inflammatory disease, or diseases having inflammatory effects, the method comprising administering to a patient an aqueous solution of at least one polyether polyol having a molecular weight of from 200 to 4000 which is dissolved in the aqueous solution in a concentration of from 0.01 to 10 mg/ml.

For treating inflammatory disease, or conditions having inflammatory components, the medicament solution of the present invention is preferably administered intravenously, intraperitoneally or intrathecally for central nervous system administration. The administration method is the same as for the conventional administration of an intravenous resuscitation fluid or administration of intrathecal or intraperitoneal therapy. The administration may be in a single dose, or in plural doses administered over a period of time.

In a most preferred aspect, the present inventors have found that low molecular weight polyethylene glycol administered in low concentration, of from 1-7 mg/ml, inhibits production and/or release of major endogenous pro-inflammatory mediators and prevents death in the experimental models of severe bacterial sepsis even when it is given 2 hours after the induction of sepsis, i.e. in a clinically relevant scenario. Furthermore, the present inventors have direct evidence that the protective effect of polyethylene glycol may be mediated through preservation of mitochondrial function, a key site of sepsis-mediated cellular dysfunction.

Embodiments of the present invention will now be described by way of example only with reference to the accompanying drawings, in which:

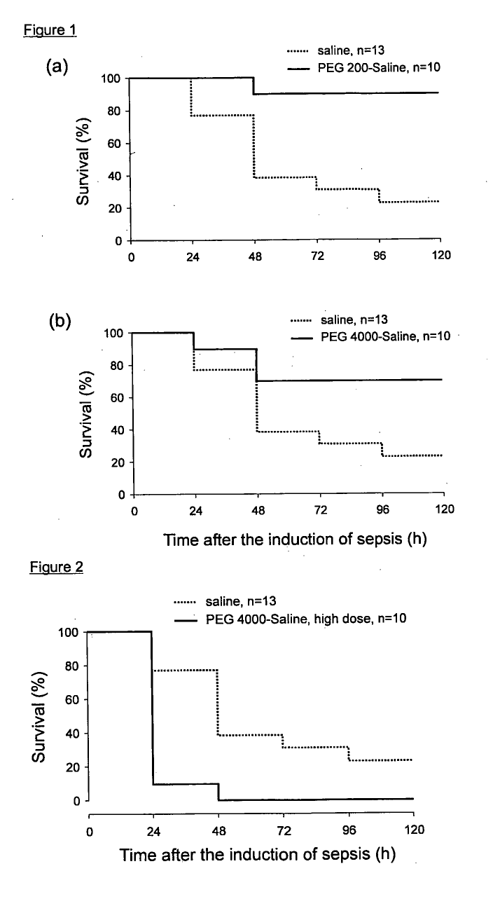

FIGS. 1 (a) and (b) show the survival of mice treated with a medicament 2 hours after the induction of sepsis and twice daily (every 12 hours) thereafter in accordance with Example 1 of the present invention and in a comparative control sample;

FIG. 2 shows the survival of mice treated with a medicament 2 hours after the induction of sepsis and twice daily (every 12 hours) thereafter in accordance with Comparative Example 1;

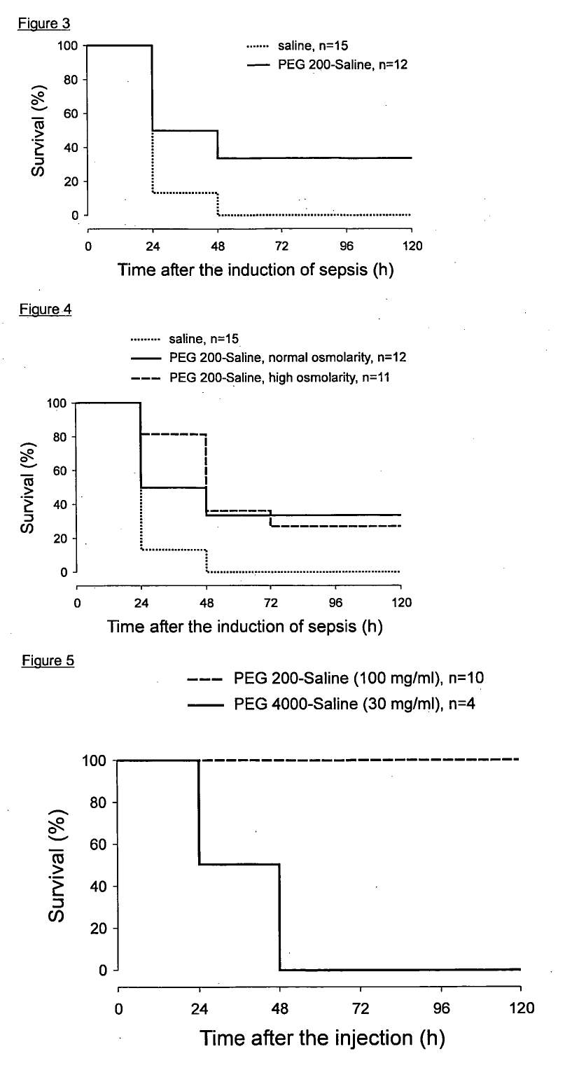

FIG. 3 shows the survival of mice treated with a medicament 2 hours after the induction of sepsis and twice daily (every 12 hours) thereafter in accordance with Example 2;

FIG. 4 shows the survival of mice treated with a medicament 2 hours after the induction of sepsis and twice daily (every 12 hours) thereafter in accordance with Example 3 of the present invention and in a comparative control sample;

FIG. 5 shows the survival of mice treated with a medicament every 12 hours in accordance with Comparative Example 2;

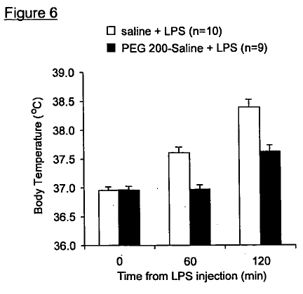

FIG. 6 shows the relationship between body temperature and time for mice treated with a medicament in accordance with Example 4 of the present invention and in a comparative control sample;

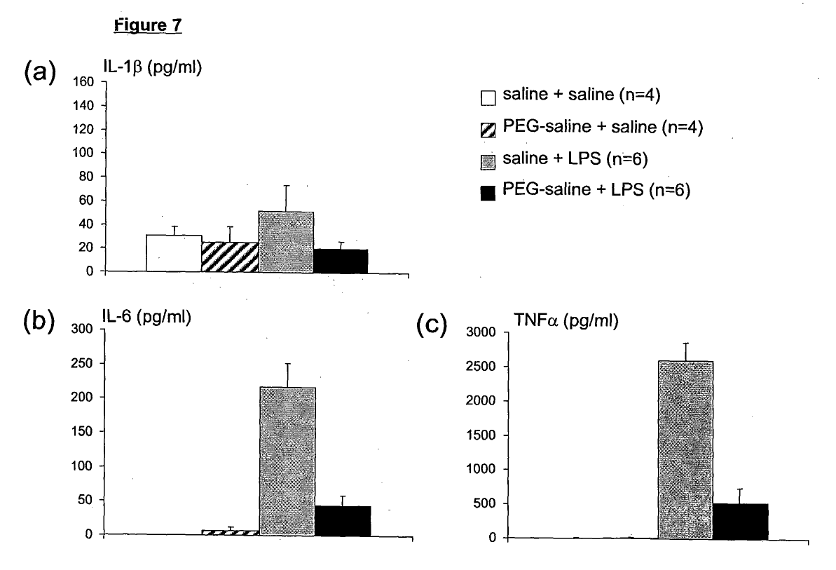

FIGS. 7 (a), (b) and (c) show the relationship between blood plasma levels of the respective cytokines and composition of the injection for rats treated with a medicament in accordance with Example 5 of the present invention and in comparative control samples; and

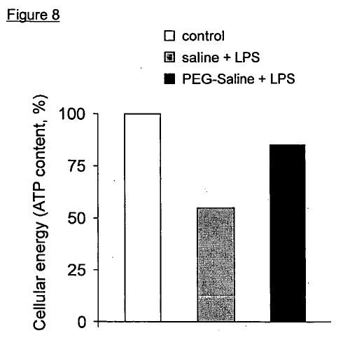

FIG. 8 shows measurements of cellular energy content in vitro after treatment of activated macrophages with a solution in accordance with Example 6 of the present invention and with a comparative control.

The present invention is predicated on the finding by the inventors that there is a marked benefit resulting from the administration of polyether polyol, in particular polyethylene glycol-saline solutions in experimental models of acute inflammation, including prevention of death in lethal inflammation/sepsis. The therapeutic application of this finding uses widely practiced, accepted means of maintaining/restoring organ function through administration of fluid, for example as a resuscitation fluid, but with the additional benefit of anti-inflammatory substance within that fluid.

The present invention therefore is based in part on the discovery of a new and unexpected medical use of a known compound, which, according to the experimental data obtained by the present inventors, has profound anti-inflammatory properties, which to the inventors' knowledge was previously unrecognised by those skilled in the art. In accordance with the present invention, the polyethylene glycol-saline solution can be administered easily, safely and effectively. The inventors believe that because polyethylene glycol has previously been recognised as being safe for human use, being used widely in foods and drugs (categorised as a “GRAS” (Generally Recognised as Safe) substance by The United States Food and Drug Administration), there is an immediate clinical opportunity to develop polyethylene glycol-saline solutions as a life-saving therapeutic intervention in critically ill individuals. Moreover, the administration of the polyether polyol-saline solutions may readily be achieved using a standard mode of care (fluid therapy) to deliver, in a resuscitation fluid, the additional anti-inflammatory benefit of the polyether polyol compounds.

The data obtained experimentally by the present inventors shows that small amounts of polyethylene glycol, dissolved in fluid used internally for resuscitation (for example an aqueous solution of one or more crystalloids, an aqueous suspension of one or more colloids, or a mixture thereof), prevents death in experimental models of severe sepsis even when it is administered 2 hours after the induction of sepsis.

The medicament of the present invention may be used for its anti-inflammatory properties for the treatment of pathophysiological states where there is acute and/or chronic release of inflammatory mediators, for example: sepsis/septic shock; acute respiratory distress syndrome; and major surgical procedures associated with major inflammatory response.

The present invention will now be described in greater detail with reference to the following non-limiting Examples.

### EXAMPLE 1

Two isotonic solutions of polyethylene glycol in compound sodium lactate solution in accordance with the present invention were prepared. A first solution had a concentration of 6.2 mg/ml of the polyethylene glycol in compound sodium lactate solution, and the polyethylene glycol had an average molecular weight of 200, and is available in commerce from the company Sigma, of Poole, UK (amongst many others). This polyethylene glycol solution is referred to hereinafter in these Examples as PEG 200-saline. A second solution had a concentration of 6.2 mg/ml of the polyethylene glycol in compound sodium lactate solution, and the polyethylene glycol had an average molecular weight of 4000, and is available in commerce from the company Sigma, of Poole, UK (amongst many others). This polyethylene glycol is referred to hereinafter in these Examples as PEG 4000-saline.

Adult mice were injected intraperitoneally with zymosan, which induces severe inflammation leading to systemic bacteraemia and endotoxaemia from gastrointestinal inflammation, with a predicted mortality of 80% by day 5.

The isotonic polyethylene glycol-saline solution was administered intraperitoneally (25 ml/kg) 2 hours after the onset of peritonitis (and twice daily thereafter, every 12 hours). This administration protocol represented both a realistic clinical and therapeutic timeframe of events. Ten mice (i.e. n=10) were administered the low molecular weight (200) polyethylene glycol-saline solution (the first solution) and another ten mice (i.e. n=10) were administered the high molecular weight (4000) polyethylene glycol-saline solution (the second solution). A further thirteen mice (i.e. n=13) were administered, as a control, with the saline solution alone, not containing any polyethylene glycol.

The results are summarised in FIGS. 1 (a) and (b), which show survival plots depicting percentage of mice alive vs. time after the onset of sepsis. Numbers in parentheses indicate sample sizes at the outset of the experiment. It was found that this administration of the isotonic polyethylene glycol-saline solution reduced mortality by 55-75% (depending on the molecular weight of polyethylene glycol). The low molecular weight (200) polyethylene glycol improved survival more than the high molecular weight (4000) polyethylene glycol.

This Example shows that administration of a polyethylene glycol-saline solution in accordance with the present invention can substantially reduce mortality in a clinically relevant model of severe inflammation and sepsis.

### COMPARATIVE EXAMPLE 1

Example 1 was repeated but with polyethylene glycol 4000-saline in a high concentration of 30 mg/ml of the polyethylene glycol in compound sodium lactate solution, which was administered to the mice (n=10) at a dose of 25 ml/kg. This had a detrimental effect, worsening the outcome of zymozan-induced sepsis, even compared to the saline control. The survival rate was less than 20% after only 24 hours, and zero after 48 hours. This is shown in FIG. 2.

### EXAMPLE 2

Experimental endotoxaemic sepsis has been shown to cause higher mortality in female animals (M K Angele et al., Vol 14 Shock pp. 81-90, 2000). Female mice were injected intraperitoneally with lethal dose (2.5 mg/kg) of E. coli endotoxin lipopolysaccharide, followed 2 hours later with fluid resuscitation of either control saline (n=15) or the low molecular weight (200) polyethylene glycol-saline solution (the first solution of Example 1) (n=12). The mice were injected intraperitoneally twice daily thereafter.

The results are summarised in FIG. 3.

All control (i.e. saline treated) mice died within 48 h after lipopolysaccharide injection, while ˜35% of polyethylene glycol 200-saline administered animals survived and completely recovered.

It should be noted that endotoxin given intraperitoneally is cleared by residual macrophages within minutes and would not be present in the peritoneal cavity at the time when the first injection of polyethylene glycol-saline is made. Thus, without being bound by theory, the inventors believe that direct neutralization of lipopolysaccharide or blockade of its interaction with the receptor by polyethylene glycol is unlikely, and that it is more feasible that polyethylene glycol-saline exerts its protective effect by counteracting inflammatory process triggered by lipopolysaccharide following absorption.

This Example provides further evidence that administration of a polyethylene glycol-saline solution in accordance with the present invention can substantially reduce mortality in lethal endotoxaemia and sepsis.

### EXAMPLE 3

Hyperosmolar fluid resuscitation has been reported previously to reduce experimental inflammation in comparison to resuscitation with solutions of normal osmolarity (Wade C E, Vol 6 (5) Crit. Care pp. 397-8, 2002). However, no difference in survival was found when sepsis-hypersensitive female mice exposed to lethal endotoxaemia (2.5 mg/kg E. coli lipopolysaccharide) were resuscitated with either isomolar (similar osmolarity as normal blood; 293 mOsmol/l, 6.2 mg/ml PEG 200) or hyperosmolar (higher osmolarity than normal blood; 318 mOsmol/l, 50 mg/ml PEG 200) polyethylene glycol-saline solution (using twice daily intraperitoneal injections). The polyethylene glycol was the same low molecular weight (200) polyethylene glycol as used in the first solution of Example 1.

The results are summarised in FIG. 4. In both isomolar and hyperosmolar polyethylene glycol-saline treated groups (n=11-12 mice), mortality was reduced by the same extent. Approximately 30% of sepsis-hypersensitive female mice treated with polyethylene glycol-saline solutions fully recovered from lethal endotoxaemia. In the control saline-treated group (n=15) no animals survived beyond the 48 hour time point. These results indicate that beneficial effect of polyethylene glycol on survival during sepsis is not due to its effects on fluid osmolarity.

### COMPARATIVE EXAMPLE 2

In this example, the toxic effects of high molecular weight polyethylene glycol at high concentrations were compared to the effects of low molecular weight polyethylene glycol at even higher concentrations.

Normal mice, which were not subjected to zymozan or lipopolysaccharide injections, were infused (25 ml/kg) with either PEG-200-saline solution at a PEG-200 concentration of 100 mg/ml or PEG-4000-saline solution at a PEG-4000 concentration of 30 mg/ml. The results are summarised in FIG. 5.

All animals treated with the PEG-200-saline solution survived and exhibited no obvious signs of sickness or discomfort.

In contrast, all animals treated with the PEG-4000-saline solution (at around a three times lower concentration than the PEG-200-saline solution) died within 48 hours after the injection. This may also be compared to Example 1 where PEG-4000 administered in a lower concentration of 6.2 mg/ml improved survival in a clinically relevant model of zymozan-induced sepsis (FIG. 1).

### EXAMPLE 4

In this Example, a PEG-200-saline solution (10% PEG-200 in a dose of 1 ml/kg) was injected intraperitoneally into rats (n=10) having had fever induced by intraperitoneal injection of E. coli endotoxin lipopolysaccharide, at an amount of 50 μg/kg bodyweight. The temperature of the rats was measured for a period of two hours. As a comparison, the temperature was correspondingly measured in rats (n=9) injected intraperitoneally with a control saline solution after having had fever induced by intraperitoneal E. coli endotoxin lipopolysaccharide administration. The results are summarised in FIG. 6. The temperature data for each time measurement are presented as mean temperature values ±standard error of the mean value.

It was found that body temperature of rats treated with both polyethylene glycol 200-saline solution and lipopolysaccharide was significantly lower than of rats injected with saline and lipopolysaccharide (p<0.05). The data demonstrate that one of the major indicators of systemic inflammation, i.e. fever, is markedly reduced following a single intraperitoneal injection of polyethylene glycol-saline in accordance with the invention.

### EXAMPLE 5

In this Example it was found that bacterial endotoxin lipopolysaccharide (LPS)-evoked production and/or release of key pro-inflammatory mediator cytokines such as interleukin-1β (IL-1β), interleukin-6 (IL-6) and tumour necrosis factor-α (TNFα) are markedly reduced by polyethylene glycol 200-saline solution (the low molecular weight polyethylene glycol used in the first solution of Example 1) treatment in rats.

In this Example, rats were injected sequentially with either (a) saline and saline (n=4); (b) the low molecular weight (200) polyethylene glycol-saline solution (10% PEG in a dose of 1 ml/kg) and saline (n=4); (c) saline and bacterial endotoxin—E. coli lipopolysaccharide (LPS, 50 μg/kg)—dissolved in saline (n=6); or (d) the low molecular weight (200) polyethylene glycol-saline solution and bacterial endotoxin—E. coli lipopolysaccharide (LPS, 50 μg/kg)—dissolved in saline (n=6). Plasma IL-1β, IL-6 and TNFα concentrations were measured after a period of 1 hour following injection of the respective solutions (either containing LPS or not). The results are summarised in FIGS. 7 (a), (b) and (c), which show data presented as means ±standard errors of the means. Numbers in parentheses indicate sample sizes.

It was found that IL-1β, IL-6 and TNFα levels in plasma of rats treated with both polyethylene glycol 200-saline solution and lipopolysaccharide were significantly lower than in plasma of rats injected with saline and lipopolysaccharide (p<0.05).

This Example shows that administration of a polyethylene glycol-saline solution in accordance with the present invention can substantially reduce the production and/or release of major pro-inflammatory cytokines during systemic inflammation evoked by bacterial endotoxins.

### EXAMPLE 6

This Example shows that polyethylene glycol effectively protects cellular function by reducing the toxic effect of bacterial endotoxin-lipopolysaccharide on cellular energy content.

In this Example, macrophages were treated in vitro with LPS in the presence or absence of polyethylene glycol 400. “Cellular energy content” was determined by measuring the amount of intracellular adenosine-5-triphosphate (ATP)— the major cellular source of energy produced by mitochondria. The results are shown in FIG. 8.

In the untreated control, the ATP intracellular concentration, representing cellular energy, was measured and set at 100% as a baseline for comparison. After treatment with saline and LPS, the ATP content was reduced to 55% of that of the control. This LPS-induced fall in intraceullar ATP concentration was markedly reduced in the presence of polyethylene glycol. In these conditions, ATP concentration was only reduced by 15%. The concentration of polyethylene glycol-saline was identical to that which improved survival in the animal studies (Example 1).

In short, these data indicate that mitochondrial (and, therefore, cellular function) can be preserved/protected by polyethylene glycol-saline administration in accordance with the invention.

In summary, these Examples in accordance with the invention show that treatment with polyethylene glycol-saline solutions markedly reduces mortality in two experimental models of severe acute systemic inflammation and sepsis. The polyethylene glycol-saline was found to protect against lethal endotoxaemia in 30% of mice and almost abolished mortality in zymozan-induced experimental peritonitis, where 80% mortality is expected. Endotoxin-induced production of pro-inflammatory cytokines was inhibited, through systemic actions of polyethylene glycol-saline. It has also been shown that mitochondrial function can be preserved/protected by polyethylene glycol-saline administration in accordance with the invention. From these data the inventors conclude, without being bound by theory, that the protective effect of polyethylene glycol-saline infusion in severe inflammatory conditions is due to inhibition of overzealous production and/or release of harmful quantities of pro-inflammatory cytokines along with protection of mitochondrial function and preservation of cellular energy.

It is believed that the present Examples in accordance with the invention demonstrate that a polyethylene glycol-saline fluid decreases production of pro-inflammatory mediators, protects cellular function and prevents death in severe sepsis. The implications of the data are far reaching, with immediate commercial and clinical applications, as would be apparent to medical practitioners.

1. An aqueous solution of at least one polyether polyol in an aqueous fluid for administration to a mammal, wherein the at least one polyether polyol has a molecular weight of from 200 to 4000 and is dissolved in the aqueous fluid in a concentration of from 0.01 to 10 mg/ml.

2. An aqueous solution of at least one polyether polyol in an aqueous fluid for administration to a mammal for use as a medicament wherein the at least one polyether polyol has a molecular weight of from 200 to 4000 and is dissolved in the aqueous fluid for in a concentration suitable for the treatment of an inflammatory disease.

3. An aqueous solution according to claim 2 wherein the concentration of the at least one polyether polyol in the aqueous solution is from 0.01 to 10 mg/ml.

4. An aqueous solution according to claim 1 wherein the concentration of the at least one polyether polyol in the aqueous solution is from 1 to 10 mg/ml.

5. An aqueous solution according to claim 1 wherein the polyether polyol comprises a single polyether polyol, or a mixture of at least two polyether polyols.

6. An aqueous solution according to claim 1 wherein the polyether polyol is a polyether glycol.

7. An aqueous solution according to claim 6 wherein the polyether polyol is polyethylene glycol.

8. An aqueous solution according to claim 7 wherein the polyethylene glycol has a molecular weight of from 200 to 1000.

9. An aqueous solution according to claim 1 wherein the aqueous fluid is an aqueous intravenous resuscitation fluid for administration to a human being or an aqueous fluid for administration to a human being for intrathecal or intraperitoneal therapy.

10. An aqueous solution according to claim 9 wherein the aqueous fluid is an aqueous solution of one or more crystalloids, an aqueous colloid suspension of one or more colloids, or a mixture of such an aqueous solution of one or more crystalloids and such a colloid suspension of one or more colloids.

11. An aqueous solution according to claim 1 wherein the at least one polyether polyol consists of the sole active therapeutic constituent of the aqueous solution.

12. An aqueous solution according to claim 11 wherein the aqueous solution consists of the at least one polyether polyol in a liquid selected from the group consisting of: a saline solution, a Ringer's lactate solution, a compound sodium lactate solution, an aqueous colloid suspension, and a mixture of two or more of those liquids.

13-34. (canceled)

35. A medicament for administration to a mammal, the medicament comprising at least one polyether polyol in aqueous solution, the at least one polyether polyol consisting of the sole active therapeutic constituent of the medicament, wherein the at least one polyether polyol has a molecular weight of from 200 to 4000 and is dissolved in an aqueous fluid selected from the group consisting of: an aqueous solution of one or more crystalloids, an aqueous colloid suspension of one or more colloids, and a mixture of such an aqueous solution of one or more crystalloids and such a colloid suspension of one or more colloids.

36. A medicament according to claim 35 wherein the at least one polyether polyol is in a concentration of from 0.01 to 10 mg/ml in the aqueous fluid.

37. A medicament according to claim 36 wherein the at least one polyether polyol is in a concentration of from 1 to 10 mg/ml in the aqueous fluid.

38. A medicament according to claim 35 wherein the polyether polyol comprises a single polyether polyol, or a mixture of at least two polyether polyols.

39. A medicament according to claim 35 wherein the polyether polyol is a polyether glycol.

40. A medicament according to claim 39 wherein the polyether polyol is polyethylene glycol.

41. A medicament according to claim 40 wherein the polyethylene glycol has a molecular weight of from 200 to 1000.

42. A medicament according to claim 35 wherein the medicament is an intravenous resuscitation fluid or a fluid for intrathecal or intraperitoneal therapy.

43. A medicament according to claim 35 wherein the aqueous fluid is selected from the group consisting of: a saline solution, a Ringer's lactate solution, a compound sodium lactate solution, an aqueous colloid suspension and a mixture of two or more of those liquids.

44. A method of treating an inflammatory disease, or a disease having inflammatory effects, the method comprising administering to a patient an aqueous solution of at least one polyether polyol having a molecular weight of from 200 to 4000 which is dissolved in the aqueous solution in a concentration of from 0.01 to 10 mg/ml.

45. A method according to claim 44, wherein the concentration of the at least one polyether polyol in the aqueous solution is from 1 to 10 mg/ml.

46. A method according to claim 44 wherein the aqueous solution is administered intravenously.

47. A method according to claim 44 wherein the aqueous solution is administered intrathecally.

48. A method according to claim 44 wherein the aqueous solution is administered intraperitoneally.

49. A method according to claim 44 wherein the patient is a human being.
